(19) United States
(12) Patent Application Publication
(10) Pub. No.: US 2010/0331913 A1
(43) Pub. Date: Dec. 30, 2010

(54) Hybrid multi-function electrode array

(76) Inventors: Alfred E. Mann, Beverly Hills, CA (US); William Vanbrooks Harrison, Valencia, CA (US)

Correspondence Address: Wong Cabello Lutsch Rutherford & Brucculeri LLP, 20333 Tomball Pkwy, Suite 600, Houston, TX, 77070, US

(21) Appl. No.: 11/261,432

(22) Filed: Oct. 28, 2005

(51) Int. Cl. A61N 1/05

(52) U.S. Cl. 607/57

(57) ABSTRACT

A hybrid electrode array includes a basal array section and a distal array section. The basal array section is configured to provide high frequency stimulation. The basal array section is configured to extend into the cochlea up to a region where electrical stimulation provides recovery for high frequency loss. The distal array section is configured to be attached to a distal tip of the basal array section and is configured to extend into the cochlear to a region where electrical stimulation provides recovery for middle to low frequency hearing loss. For progressive hearing loss treatment, the distal array section is not activated during initial stages of hearing loss allowing the patient to rely on a combination of acoustic stimulation and high frequency stimulation provided by the basal array section. As hearing loss progresses, the distal array section is selectively activated to treat lower frequency hearing loss using lower frequency stimulation.

### BACKGROUND

This document relates to implantable electrode arrays for use with a cochlear stimulation (or cochlear prosthesis) system for the treatment of hearing loss.

Generally, there are two types of hearing loss: conductive and sensorineural. Conductive hearing loss occurs when the normal mechanical pathways for sound to reach the hair cells in the cochlea are impeded, for example, by damage to the ossicles. Sensorineural hearing loss occurs when the hair cells in the cochlea, which are needed to transduce acoustic signals into auditory nerve impulses, are either absent or destroyed.

Conductive hearing loss typically may be treated with the use of a hearing aid system, which amplifies sound so that acoustic information can reach the cochlea and the hair cells, or through surgical procedures. Hearing aids, however, are not effective for treating sensorineural hearing loss, no matter how loud the acoustic information is amplified, because the hair cells in the cochlea are either absent or destroyed.

Sensorineural hearing loss typically may be treated with a cochlear stimulation (or cochlear prosthesis) system, such as one described in U.S. Pat. Nos. 5,938,691 and 6,219,580, which are incorporated herein by reference. A cochlear stimulation system operates by direct electrical stimulation of the ganglia of the auditory nerve cells, whereby the defective cochlear hair cells that normally transduce acoustic energy into electrical activity in such nerve cells are bypassed. When stimulated, the ganglia, also referred to as ganglion cells, send nerve impulses to the brain via the auditory nerve, leading to the perception of sound in the brain. Conventional cochlear stimulation systems generally include an electrode array, which is implanted into the cochlea. The electrode array receives electrical stimuli, which represents converted auditory information, and transmits the electrical stimuli to the ganglion cells, and thereby to the auditory nerve fibers.

A large segment of the hearing-impaired population exhibits sensorineural hearing loss relative to high frequency sounds, but is still able to transduce middle-to-lower frequency sounds, with or without a conventional hearing aid, through functioning hair cells in the cochlea. For this segment of the population, hearing loss treatment typically includes use of an implantable cochlear stimulation system that electrically stimulates the ganglion cells responsible for sensing higher frequency sounds and use of a hearing aid (or no hearing aid) for sensing middle-to-low frequency sounds.

Because the ganglion cells responsible for sensing higher frequency sounds are generally located in or near the basal end of the cochlea (the end of the cochlea nearest the round window membrane), in conventional cochlear stimulation systems, the electrode array is surgically inserted within the cochlea a sufficient depth to be near such cells, but not deep enough to interfere with the functioning of the hair cells deeper within the cochlea that aid in sensing middle-to-low frequency sounds. Surgical insertion of the electrode array, however, has potential risks. Once such risk is the complete or partial loss of the remaining residual acoustic hearing, e.g., the middle-to-low frequency sounds. If that occurs, then treatment typically requires total electrical stimulation. Therefore, a second or third surgery is needed to replace the electrode array (and possibly other components of the cochlear stimulation system) with a longer array to enable electrical stimulation of the ganglion cells responsible for sensing middle-to-low frequency sounds.

### SUMMARY

The present inventors recognized that conventional cochlear stimulation systems and techniques used for treating high frequency precipitous hearing loss tend to utilize short electrode arrays that are confined within the cochlea to be near high-frequency sensing ganglion cells, and therefore are unable to provide electrical stimulation in middle-to-low frequency ranges unless a longer electrode array is surgically inserted, typically requiring a second or third surgery. Consequently, the present inventors developed a hybrid multi-function electrode array so as to enable electrical stimulation at any desired frequency. The electrode array may include a basal array section, which may provide stimulation in the high frequency range, and a distal array section, which may provide stimulation in the middle-to-low frequency range.

Implementations of the hybrid multi-function electrode array and associated techniques for treating hearing loss described here may include various combinations of the following features.

In one implementation, a hybrid multi-function electrode array may include a basal array section for insertion into a basal region of the scala tympani duct of a cochlea and configured to provide high frequency stimulation, and a distal array section for insertion into at least one of a middle region and an apical region of the scala tympani duct of the cochlea and configured to provide middle to low frequency stimulation. The basal array section may also include a first flexible carrier having a first end and a second end, and electrodes carried on the first flexible carrier. The distal array section includes second flexible carrier having a first end and a second end, wherein the first end of the second flexible carrier is coupled to the second end of the first flexible carrier, and electrodes carried on the second flexible carrier. The hybrid multi-function electrode array may also include wires, each wire passing through the first end of the first flexible carrier to a corresponding one of the plurality of electrodes carried on the first flexible carrier and the second flexible carrier.

The hybrid multi-function electrode array may have the following dimensions. In one implementation, the cross sectional area of the first flexible carrier is greater than a cross-sectional area of the second flexible carrier. In another implementation, the cross sectional area of the first end of the first flexible carrier is larger than a cross-sectional area of the second end of the first flexible carrier. In yet another implementation, the first flexible carrier has a length of about 13.5 mm, a width of less than or equal to 1.0 mm, and a thickness of less than or equal to 1.0 mm, and the second flexible carrier has a length of about 9 mm, a width of less than or equal to 0.5 mm, and a thickness of less than or equal to 0.5 mm.

In another implementation, the hybrid multi-function electrode array may include a first flexible carrier having electrodes on one or more surfaces of the first flexible carrier and configured for insertion into a basal region of the cochlea to provide high frequency stimulation, and a second flexible carrier having electrodes on one or more surfaces of the second flexible carrier and configured for insertion into a middle or an apical region of the cochlea or both to provide middle to low frequency stimulation, wherein an end of the second flexible carrier is attached to an end of the first flexible carrier. Additionally, wires carried within the first flexible carrier and the second flexible carrier may be connected to a corresponding one of the electrodes carried on the first flexible carrier and the second flexible carrier. The plurality of wires may be configured to be coupled to an implantable cochlear stimulator for receiving electrical stimuli.

In one implementation, techniques for treating hearing loss using the hybrid multi-function electrode array may include inserting an electrode array into a cochlea such that a basal array section of the electrode array is positioned within a basal region of the cochlea and a distal array section of the electrode array is positioned within a middle region or an apical region of the cochlea or both, generating a high frequency electrical stimulus representative of a high frequency content of a sensed acoustic signal, applying the high frequency electrical stimulus to the basal region of the cochlea through the basal array section of the electrode array to compensate for high frequency hearing loss, generating a middle-to-low frequency electrical stimulus representative of a middle-to-low frequency content of the sensed acoustic signal, and applying the middle-to-low frequency electrical stimulus to the middle region or the apical region of the cochlea or both through the distal array section of the electrode array to compensate for middle-to-low frequency hearing loss.

The hybrid multi-function electrode arrays described here may provide several advantages. For example, in treating the initial stages of hearing loss (typically high frequency), the hybrid multi-function electrode array is surgically inserted into the patient's cochlea and the basal array section is activated, but the distal array section is not, thereby allowing the patient to rely on a combination of high frequency electrical stimulation provided by the basal array section and acoustic stimulation typically provided by a hearing aid. As the hearing loss progresses to middle-to-low frequencies, the distal array section may be activated in phases, thereby allowing the patient to rely on middle-to-low frequency electrical stimulation. By using the hybrid electrode array, this treatment may be accomplished with a single surgery. In contrast, by using conventional electrode arrays, which typically only provide high frequency coverage, a second or third surgery may be required in order to insert a longer electrode array that can provide the required frequency coverage to treat the patient's hearing loss. This results in substantial medical cost savings and minimizes the risk of further hearing loss caused by repeated surgeries.

The details of one or more implementations are set forth in the accompanying drawings and the description below. Other features and advantages will be apparent from the description and drawings, and from the claims.

### DESCRIPTION OF DRAWINGS

FIG. 1 is a functional schematic diagram of the ear, showing the manner in which an implantable cochlear stimulation system utilizing a hybrid multi-function electrode array may be implemented.

FIGS. 2a and 2b illustrate an implementation of the hybrid multi-function electrode array.

FIGS. 3a and 3b illustrate one method of inserting an implementation of the hybrid multi-function electrode array into the cochlea.

Like reference symbols in the various drawings indicate like elements.

### DETAILED DESCRIPTION

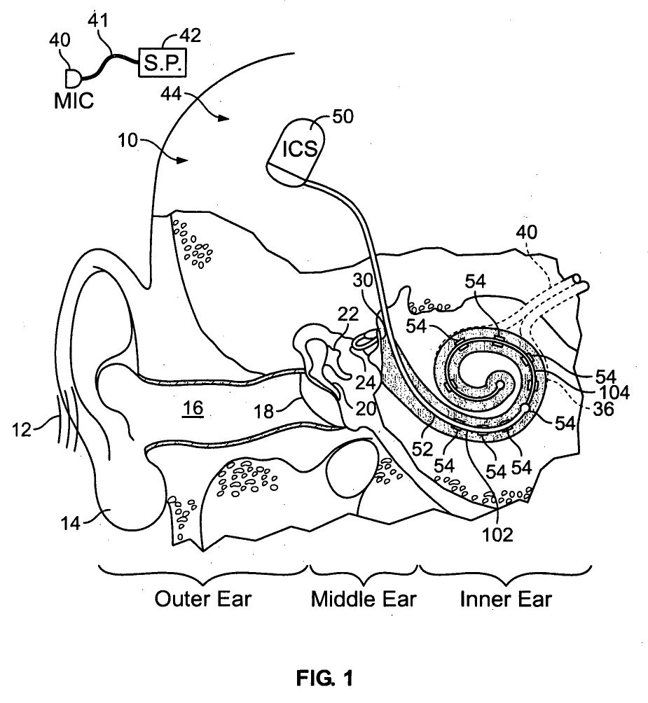

FIG. 1 depicts certain major components of the human ear and illustrates the manner in which a cochlear stimulation system utilizing a hybrid multi-function electrode array (hereinafter, “hybrid electrode array”) may be implemented. To better understand the various implementations, it is helpful to briefly review the normal operation of a fully functional ear.

The outer ear includes the auricle 14 and the ear canal 16. An acoustic signal, or sound wave, represented by the short parallel lines 12, is received by the auricle 14 and funneled into the ear canal 16. At the end of the ear cannel 16 is the ear drum 18, or in medical terms, the tympanic membrane 18. In a person who is not significantly hearing impaired, the received acoustic signal 12 causes the tympanic membrane 18 to vibrate. The vibration is coupled through three tiny bones, the malleus (“hammer”) 20, the incus (“anvil”) 22 and the stapes (“stirrup”) 24, to the fenestra 30.

In anatomical terms, the fenestra comprises an opening resembling a window with two parts. The first part, the fenestra ovalis, or oval window, is the opening between the middle ear and the vestibule of the inner ear. It is closed by a membrane to which the stapes is attached. The second part, the fenestra rotunda, or round window, is the opening between the scala tympani duct of the cochlea 36 and the middle ear. The round window is also closed by a membrane, which for purposes of the present application, may be referred to as the round window membrane. For purposes of the present application, the function of both the oval window and round window may be represented by the fenestra membrane 30.

The bones of the middle ear serve to filter and amplify the perceived acoustic signal 12, which cause the fenestra membrane 30 to articulate, or vibrate. The vibration of the fenestra membrane 30 causes the fluid contained within the cochlea 36 to move. This fluid motion, in turn, activates tiny hair cells that line the inside of the cochlea 36. Activation of the hair cells causes appropriate nerve impulses to be transferred through the spiral ganglion cells and auditory nerve 46 to the brain, which perceives the nerve impulses as sound.

The spiral ganglion cells that are responsible for the perception of high frequency sounds are generally located at the basal end of the cochlea 36, i.e., that end of the cochlea closest to the fenestra membrane 30, while the spiral ganglion cells 46 that are responsible for the perception of middle-to-low frequency sounds are generally located at the middle to apical region of the cochlea 36. For those persons who suffer from high frequency hearing loss, the hair cells in the basal region of the cochlea 36 are ineffective or otherwise damaged to the point where it is not possible to activate them. Likewise, for those persons who suffer from middle-to-low frequency hearing loss, the hair cells in the middle to apical region of the cochlea 36 are ineffective or otherwise damaged.

A cochlear stimulation system including a hybrid electrode array 52 may be used to effectively treat such persons, both those suffering from high frequency hearing loss and those suffering from middle-to-low frequency hearing loss. A cochlear stimulation system may include microphone 40, speech processor (SP) 52, implantable cochlear stimulator (ICS) 50, and hybrid electrode array 52. The microphone may be coupled, through communication link 41, to the SP 52, which may be external or implanted. The communication link 41 may be an insulated copper wire, a radio frequency (RF) link, or any other data link capable of communication sensed acoustic signals to the SP 42.

The SP 42 is coupled to the ICS 50 through communications link 44, which may be an inductive coupling link, an insulated copper wire, or any other data link capable of communicating electrical or data signals from the SP 42 to the ICS 50. Typically, communications link 44 is a transcutaneous data link that allows power and control signals to be sent from the SP 50 to the ICS 50. Likewise, data and status signals may be transmitted from the ICS 50 to the SP 42. A transcutaneous data link may be accomplished by an internal antenna coil within the ICS 50, and an external antenna coil within the SP 42. In operation, the external antenna coil may be positioned so as to be aligned over the location where the internal antenna coil is implanted, allowing such coils to be inductively coupled to each other, thereby allowing data (e.g., the amplitude and polarity of a sensed acoustic signal) and power to be transmitted from the SP 42 to the ICS 50.

The hybrid electrode array 52 may include two sections: the basal array section 102 and the distal array section 104. Both sections 102, 104 may have a plurality of spaced apart electrodes 54 located within or on the hybrid electrode array 52, which may form one or more cochlea stimulating channels, each typically associated with an individual electrode 54 or a pair or group of electrodes 54. The hybrid electrode array 52 may be inserted directly through a slit made in the round window of the membrane 30. The basal array section 102 may be inserted into the basal region of the cochlea 36 where spiral ganglion cells that perceive high frequency sounds are located, i.e., that region of the cochlea 36 where electrical stimulation provides recovery for high frequency hearing loss. The basal region may extend as low as 700 hertz and may require up to 240 degrees of insertion depth for full coverage of the basal region. The basal array section 102 may be designed to be atraumatic and to provide chronic use. The distal array section 104 may be long and thin, and may be inserted into the middle to apical regions of the scala tympani duct of the cochlea 36 where spiral ganglion cells that perceive middle-to-low frequency sounds are located, i.e., that region of the cochlea 36 where electrical stimulation provides recovery for middle-to-low frequency hearing loss. The distal array section 104 may be designed to be atraumatic and to reside in the scala tympani duct of the cochlea 36 away from the basilar membrane to allow the basilar membrane to vibrate freely in response to acoustic stimulation.

In operation, the acoustic signals 12 sensed by the microphone 40 are amplified and processed by SP 42. The SP 42 generates appropriate control signals that are communicated to the ICS 50 through communications link 44. The ICS 50 uses the control signals to selectively generate electrical stimuli and to apply the electrical stimuli to the spiral ganglion cells through the electrodes 54 on the hybrid electrode array 52 to enhance the hearing of high-to-low frequency sounds. Such electrical stimuli bypass the defective hair cells in the cochlea and directly activate the nerves within the of the spiral ganglion 46, causing nerve impulses to be transferred to the brain, where they may be perceived as sounds.

In cases where the user has high-frequency hearing loss, the sounds sensed by the microphone 40 are processed and filtered to separate out the high frequency sounds. These high frequency sounds are then converted to high-frequency control signals, which are used by the ICS 50 to generate appropriate electrical stimuli. The ICS 50 then applies the electrical stimuli to the electrodes 54 on the basal array section 102, which is positioned in the basal region of the scala tympani duct of the cochlea 36. The electrodes 54 on the distal array section 102 are not activated and do not receive any electrical stimuli. The electrical stimuli bypass the defective hair cells in the basal region of the cochlea 36 and directly activate the nerves within the of the spiral ganglion 46, causing nerve impulses to be transferred to the brain, where they may be perceived as high frequency sounds. The other hair cells in the cochlea 36, i.e., those in the middle and apical regions of the scala tympani duct, retain their functionality because these hair cells are able to sense the fluid waves set up by vibrations of the fenestra membrane 30 corresponding to middle-to-low frequency sounds. Hence, the user is able to sense high frequency sounds through the ICS 50 portion of the system, and is able to sense lower frequency sounds through the normal hearing processes of the ear (or through the use of a hearing aide).

If the user's hearing loss progresses, the electrodes 54 on the distal array section 104 may be activated in phases to make up for any additional middle-to-low frequency hearing loss by providing electrical stimuli to the spiral ganglion cells 46 associated with the middle and apical regions of the scala tympani duct of the cochlea 36. If the user loses all acoustic hearing, the entire hybrid electrode array 52 is activated, i.e., the electrodes 54 on both the basal array section 102 and the distal array section 104 are activated, and functions in a manner similar to a conventional cochlear stimulation system. Thus, the user need not be subjected to a second or third surgery to treat the user's progressive hearing loss.

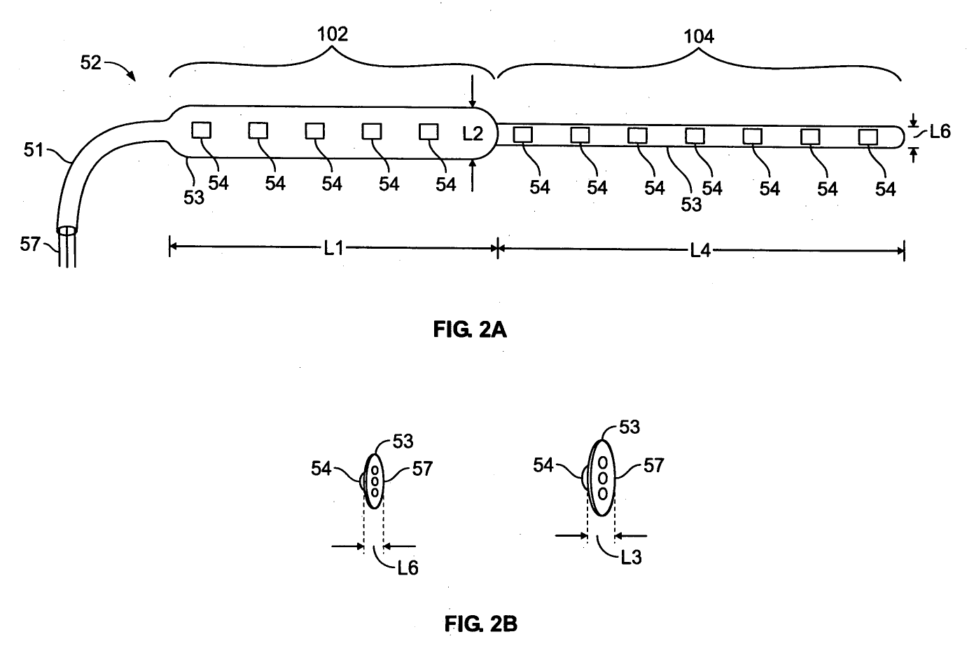

FIGS. 2a and 2b illustrate an implementation of the hybrid electrode array 52. The hybrid electrode array 52 includes a basal array section 102 and a distal array section 104. The distal array section 104 is thinner than the basal array section 104 and is attached to the distal end of the basal array section 102. The basal end of the basal array section 102 may be thicker than the distal end of the basal array section 102 so that, e.g., upon insertion of the array 52 into the cochlea 36, fluid within the cochlea may be retained. Additionally, the basal end of the basal array section 102 may include a plurality of flaps, or tines, protruding out from the body of the hybrid electrode array 52 so that, e.g., upon insertion of the array 52 into the cochlea 36 the plurality of flaps prevent the hybrid electrode array 52 from slipping out of the cochlea 36.

Both the basal array section 102 and the distal array section 104 include a plurality of spaced-apart electrode contacts 54 that are carried on a suitable flexible carrier 53, which may include a metal carrier covered with a polymer material. Each electrode contact 54 is electrically connected to at least one wire 57 that is embedded within the flexible carrier 53, and within the lead 51. The ICS 50 (not shown) provides electrical stimuli through wires 57 to selected ones of the electrode contacts 54. Alternatively, a mechanical or other transducer may also be included on the hybrid electrode array 52.

The flexible carrier 53 maybe flat and thin with the portion of the flexible 53 comprising the distal array section 104 being thinner than portion of the flexible carrier 53 of the basal array section 102. The electrode contacts 54 typically reside along one surface of flexible carrier 53. In this implementation, the electrode contacts 54 are on a medial side of the flexible carrier 53, i.e., the electrodes 54 are on the same side of the flexible carrier 53. Thus, when the hybrid electrode array 52 is inserted into the cochlea 36, the medial side of the array sections 102, 104 may be positioned to face the modiolar wall of the cochlea 36, where the ganglion cells are located. Such positioning places the electrode contacts 54 closer to the modiolar wall, thereby allowing electrical stimulation of the ganglion cells to occur more efficiently, i.e., with less power. Depending on design requirements, one or more of the electrode contacts 54 may be placed on other surfaces of the flexible carrier 53 or the electrode contacts 54 may be shaped into bands that encircle the flexible carrier 53 and still perform its intended function of stimulating the ganglion cells of the cochlea 36.

In this implementation, the basal array section 102 may have a length L1 of about 13.5 mm±1 mm, and has a width L2 of less than or equal to 1.0 mm, and has a thickness L3 of less than or equal to 1.0 mm. The distal array section 104 may have a length L4 of about 9 mm±1 mm, and has a width L5 of less than or equal to 0.5 mm, and has a thickness L6 of less than or equal to 0.5 mm. These dimensions are only exemplary, and the actual dimensions may vary as needed depending on the anatomy of particular patients. In particular, the total length of the hybrid electrode array 52 may be as long as 22.5 mm±2 mm, measured from the beginning of the basal array section to the tip of the distal array, with the total length of the active area of the electrode array 52 may be as long as 14 mm±1 mm, and the number of electrode contacts may vary from as few as one or two to as many as thirty-two or more depending on the desired resolution of the electrical stimulation. In this implementation, there are eighteen electrode contacts-sixteen active electrode contacts and two inactive (dummy) electrode contacts. The inactive (dummy) electrode may act as visual insertion depth indicators for the implanting surgeon. Due to the 1.0 mm cross-section of the basal array section 102, the cochleostomy size can be reduced from about 1.6 mm by 1.2 mm using conventional electrode arrays to 1.0 mm round.

In this implementation, the hybrid electrode array section 52 has a uniform pitch. The uniform pitch can be calculated by dividing the active array length by the number of active electrode contacts minus one. Given the active array length is 14 mm±1 mm and the number of active electrode contacts is 16, then the pitch is 14 mm/15 mm or 0.93 mm pitch. The length of the active area of the basal array section 102 is then the number of active basal electrode contacts minus one times the pitch, or (10−1)*0.93, which equals to about 8.4 mm. The length of the active area of the distal array section 104 basal electrode contacts is then the number of active distal electrode contacts minus one times the pitch, or (6−1)*0.93, which equals to about 4.7 mm. Thus, the total length of the active area of the hybrid electrode array is about 14 mm (or 8.4+4.7+0.93).

The hybrid electrode array 52 may be made by attaching the electrodes 54 made from precious, biocompatible material, such as platinum or its alloys to a metal carrier of the flexible carrier 53 made from a non-toxic but chemically-active metal, such as iron. Resistance welding may be used to attached the electrodes 54 to the metal carrier. Resistance welding typically provides a secure attachment of the electrodes 54 to the metal carrier without causing a deep fusion of the two materials being attached. The resulting shallow fusion contact, in turn, allows clean exposed electrode surface areas to be formed when the metal carrier is eventually chemically etched away. Other methods of attachment that result in shallow fusion of the electrodes 54 and the metal carrier may also be used in lieu of resistance welding. After the electrodes are attached to the metal carrier, a wiring system, with at least one wire 57, may be connected. Then, polymer molding of the flexible carrier 53 may be begun. After completion of the molding process, the metal carrier of the flexible carrier 53 may be chemically etched away using a mixture of diluted acids, such as HNO.sub.3 and HCl. The precious metal electrodes 54 and polymer are immune to the acid and remain in their intact, unaltered shape, and thereby provide the electrode array structure.

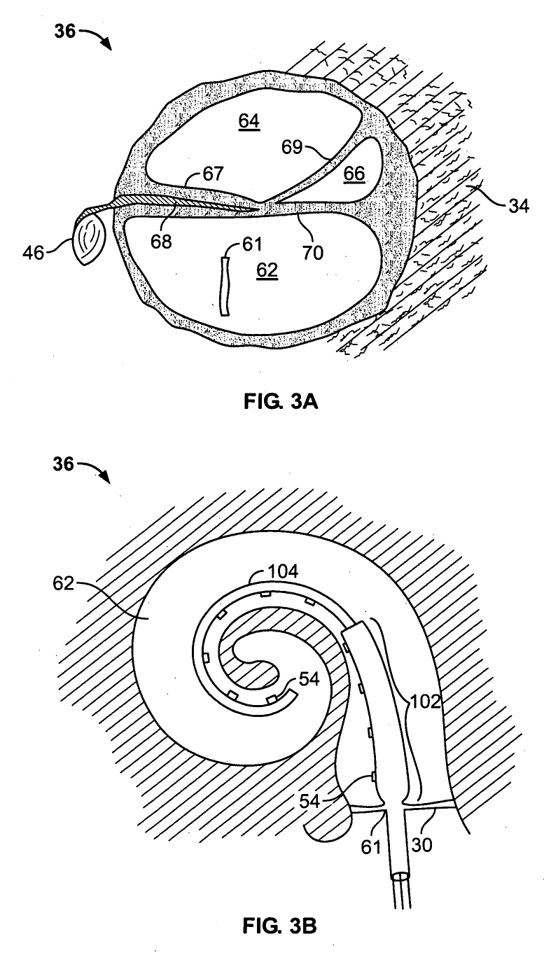

FIGS. 3a and 3b illustrate one method of inserting the hybrid electrode array 52 into the cochlea 36, which includes three parallel ducts: the scala tympani 62, the scala vestibuli 64, and the cochlear duct 66. Cochlear bony tissue 34 resides on the lateral side (the right side as drawn in FIG. 3) of the cochlea 36. Spiral ganglion cells 46 reside on the medial side (the left side as drawn in FIG. 3) of the cochlea 36. Separating the three ducts are various membranes and other tissue. The ossicous spiral lamina 67 separates the scala vestibule duct 64 from the scala tympani duct 62. Near the lateral side, which is where the cochlear duct 66 is located, the basilar membrane 70 separates the scala tympani duct 62 from the cochlear duct 66; and the vestibular (Reissner's) membrane 69 separates the scala vestibuli duct 64 from the cochlear duct 66. Many of the hair cells that are vibrated by fluid motion within the cochlea are located in or near the basilar membrane 70 and vestibular membrane 69. Nerve fibers 68, embedded within the spiral lamina 67 connect the hair cells with the spiral ganglion cells 46.

The hybrid electrode array 52 may be inserted into the scala tympani duct 62 through a narrow slit 61 made in the round window of the fenestra membrane 30. The hybrid electrode array 52 may be inserted through the slit 61 until the distal array section 104 reaches the middle to apical regions of the scala tympani duct 62, where electrical stimulation provides recovery for middle-to-low frequency hearing loss, and the basal array section 102 reaches the basal region of the scala tympani duct 62, where electrical stimulation provides recovery for high frequency hearing loss. The distal array section 104 may be inserted in the scala tympani duct 62 away from the basilar membrane 70 in order to allow the basilar membrane 70 to vibrate freely in response to acoustic stimulation. This allows a person with only high frequency hearing loss to still sense middle-to-low frequency sounds through the normal hearing processes of the ear (or through the use of a hearing aid). But as the person's hearing loss progresses, the distal array section 104 may be activated in phases to make up for any additional middle-to-low frequency hearing loss by providing electrical stimuli to the spiral ganglion cells 46 associated with the middle and apical regions of the scala tympani duct 62.

Through this type of insertion, the body of the hybrid electrode array 52, particularly the body of the basal end of the basal array section 102, effectively plugs the narrow slit 61 so that the fluid normally present within the scala tympani duct 62 may be retained so that the normal hearing processes may continue through the remaining portions of scala tympani duct 62, e.g., the middle and apical portions. Moreover, with an hybrid electrode array 52 having a plurality of flaps protruding out from the body of the basal end of the hybrid electrode array 52, the flexible flaps, once passed through the slit 61, may prevent the electrode array 52 from slipping out of the slit 61. Other methods of inserting an electrode array into the scala tympani duct 62 may also be used to insert the hybrid electrode array 52. For example, instead of inserting the hybrid electrode array 52 into the scala tympani duct 62 through the round window of the fenestra membrane 30, the hybrid electrode array 52 may be inserted into the scala tympani through a slit made near the round window of the fenestra membrane 30. This allows the fenestra membrane 30 to more effectively perform its intended function during normal hearing, i.e., vibrating in response to sensed acoustic signals, and setting up fluid waves with the fluid held within the scala tympani duct 62, which may activate the hair cells in the middle and apical regions of the cochlea 36.

A number of implementations have been described. Other implementations may include different or additional features. For example, other configurations of the hybrid electrode array may be realized and the array may provide more than two functions, i.e., three or more. The basal array section may be any conventional short electrode array typically inserted into the basal region of the cochlea to which a thinner distal array section may be attached. Accordingly, other implementations are within the scope of the following claims.

1. A cochlear electrode array comprising:
a basal array section comprising a first flexible carrier having a first cross sectional area constant along its entire length for insertion into a basal region of the scala tympani duct of a cochlea and configured to provide high frequency stimulation; and
a distal array section comprising a second flexible carrier having a constant second cross sectional area constant along its entire length for insertion into at least one of a middle region and an apical region of the scala tympani duct of the cochlea and configured to provide middle to low frequency stimulation,
wherein the first cross sectional area is greater than the second cross sectional area.

2. The cochlear electrode array of claim 1, wherein the basal array section further comprises:
a first end and a second end; and
a plurality of electrodes carried on the first flexible carrier.

3. The cochlear electrode array of claim 2, wherein the distal array section further comprises:
a first end and a second end, wherein the first end of the second flexible carrier is coupled to the second end of the first flexible carrier; and
a plurality of electrodes carried on the second flexible carrier.

4. The cochlear electrode array of claim 3 further comprising a plurality of wires, each wire passing through the first end of the first flexible carrier to a corresponding one of the plurality of electrodes carried on the first flexible carrier and the second flexible carrier.

5-6. (canceled)

7. The cochlear array of claim 1, wherein the first flexible carrier has a length of about 13.5 mm, a width of less than or equal to 1.0 mm, and a thickness of less than or equal to 1.0 mm, and the second flexible carrier has a length of about 9 mm, a width of less than or equal to 0.5 mm, and a thickness of less than or equal to 0.5 mm.

8. A hybrid electrode array for insertion into the cochlea, the array comprising:
a first flexible carrier having a first cross sectional area constant along its entire length and having a plurality of electrodes on one or more surfaces of the first flexible carrier and configured for insertion into a basal region of the cochlea to provide high frequency stimulation; and
a second flexible carrier having a first cross sectional area constant along its entire length and having a plurality of electrodes on one or more surfaces of the second flexible carrier and configured for insertion into at least one of a middle and an apical region of the cochlea to provide middle to low frequency stimulation,
wherein an end of the second flexible carrier is attached to an end of the first flexible carrier, and
wherein the first cross sectional area is greater than the second cross sectional area.

9. The hybrid electrode array of claim 8 further comprising a plurality of wires carried within the first flexible carrier and the second flexible carrier, each wire connected to a corresponding one of the plurality of electrodes carried on the first flexible carrier and the second flexible carrier.

10. The hybrid electrode array of claim 9, wherein the plurality of wires are configured to be coupled to an implantable cochlear stimulator for receiving electrical stimuli.

11. (canceled)

12. The hybrid electrode array of claim 10, wherein the first flexible carrier has a length of about 13.5 mm, a width of less than or equal to 1.0 mm, and a thickness of less than or equal to 1.0 mm, and the second flexible carrier has a length of about 9 mm, a width of less than or equal to 0.5 mm, and a thickness of less than or equal to 0.5 mm.

13-21. (canceled)

22. A hybrid electrode array for insertion into the cochlea, the array comprising in series:
a first carrier having a plurality of electrodes on one or more surfaces of the first carrier, the first carrier having a first cross sectional area constant along its entire length; and
a second carrier having a plurality of electrodes on one or more surfaces of the second carrier, the second carrier having a second cross sectional area constant along its entire length,
wherein the first cross sectional area is greater than the second cross sectional area, and
wherein the plurality of electrodes of the first carrier are configured to receive electrical stimuli representative of acoustic sounds of a first frequency range, and wherein the plurality of electrodes of the second carrier are configured to receive electrical stimuli representative of acoustic sounds of a second frequency range.

23. The hybrid electrode array of claim 22 further comprising a plurality of wires carried within the first carrier and the second carrier, each wire connected to a corresponding one of the plurality of electrodes carried on the first carrier and the second carrier.

24. The hybrid electrode array of claim 23, wherein the plurality of wires are configured to be coupled to an implantable cochlear stimulator for receiving electrical stimuli.

25. The hybrid electrode array of claim 22, wherein the first carrier has a length of about 13.5 mm, a width of less than or equal to 1.0 mm, and a thickness of less than or equal to 1.0 mm, and the second carrier has a length of about 9 mm, a width of less than or equal to 0.5 mm, and a thickness of less than or equal to 0.5 mm.
